(19) United States
(12) Patent Application Publication
(10) Pub. No.: US 2005/0250130 A1
(43) Pub. Date: Nov. 10, 2005

(54) Method for detecting location of probe bead in capillary bead array

(75) Inventor: Osamu Kogi, Tokyo (JP)

Correspondence Address: Reed Smith LLP, Suite 1400, 3110 Fairview Park Drive, Falls Church, VA, 22042-4503, US

(73) Assignee: Hitachi Software Engineering Co., Ltd.

(21) Appl. No.: 11/041,262

(22) Filed: Jan. 25, 2005

(30) Foreign Application Priority Data
May 7, 2004 (JP) 138309/2004

(51) Int. Cl. C12Q 1/68; G01N 33/48; G01N 33/50; G01N 33/551

(52) U.S. Cl. 435/6; 702/19; 436/524

(57) ABSTRACT

It is an object to provide a method for accurately identifying the order of probe beads, namely, the types of probes bound to the probe beads, by readily and precisely detecting the locations of all the beads in a capillary bead array using a fluorescence reading apparatus. It is also an object to significantly improve the reliability of results of biochemical or immunological inspection in which a capillary bead array is used. Probe beads 102 are arranged in a capillary 101. A solution that includes fluorescence-labeled target biomolecules is introduced into the capillary 101. A fluorescence image that includes the fluorescence spot 108 of a probe bead 105 that captured the biomolecules is obtained by the fluorescence reading apparatus. A solution that includes fluorescent material for staining all the beads is introduced into the capillary 101. A fluorescence image that includes the fluorescence spot area 114 of all the probe beads 113 is obtained by the fluorescence reading apparatus. The location and the order of the probe bead 105 that has captured the biomolecules is accurately determined by overlaying the fluorescence image that includes the fluorescence spot 108 of the probe bead that has captured the biomolecules and the fluorescence image that includes the fluorescence spot area 114 of all the probe beads 113.

### BACKGROUND OF THE INVENTION

1. Field of the Invention

The present invention relates to a method for detecting the locations of particulate beads, and especially to a method for detecting the locations of beads in a capillary bead array in which the beads are arranged in a capillary formed on soft resin, for example.

2. Background Art

Concerning a capillary bead array in which probe beads bound to probes for detecting biomolecules are arranged in a capillary formed on soft resin, for example, the following JP Patent Publication (Kokai) No. 2000-346842 A is cited as prior art relating to the confirmation of bead locations. JP Patent Publication (Kokai) No. 2000-346842 A discloses a method by which marker beads whose color or size is different from those of probe beads are disposed at intervals of a predetermined number of the probe beads, as a method for confirming the locations of the probe beads arranged inside a capillary. In this case, the marker beads may or may not be bound to the probes. In accordance with the locations of the marker beads as marks, the order of the probe beads, namely the types of the probes bound to the beads, can be determined. The present method is effective when the locations of the beads are confirmed by visual observation mainly using an optical microscope, for example.

By contrast, in the capillary bead array, a fluorescence detection method can be applied as a highly sensitive detection method for biomolecules. If the fluorescence detection method is applied, a sample solution that includes fluorescence-labeled target biomolecules is introduced into the capillary bead array. The biomolecules are captured by probe beads bound to probes that have affinity with the biomolecules. Only those probe beads that have captured the fluorescence-labeled target biomolecules emit fluorescence. Thus, when the fluorescence of the probe beads in a capillary bead array is detected using a fluorescence reading apparatus, only those beads that captured the fluorescence-labeled biomolecules are detected. It is very difficult to accurately know the locations and the number of these probe beads, since probe beads that do not emit fluorescence are not detected. This means that it is impossible to accurately know the order of the probe beads that emit fluorescence; namely, the types of the probes bound to the probe beads.

As mentioned above, when the fluorescence detection method is applied to a capillary bead array, it is very difficult to confirm the order of the probe beads, namely, the types of probes, using only the method for confirming the locations of the beads as disclosed in JP Patent Publication (Kokai) No. 2000-346842 A. Thus, a method is necessary by which the locations of all the beads may be detected, and by which the order of the probe beads, namely, the types of the probes, may be accurately identified in the capillary bead array using a fluorescence reading apparatus.

### SUMMARY OF THE INVENTION

In the aforementioned capillary bead array disclosed in JP Patent Publication (Kokai) No. 2000-346842 A, when the probe beads are subjected to a reaction with the fluorescence-labeled target biomolecules, a mixture of probe beads that emit fluorescence and probe beads that do not emit fluorescence is produced. In this case, if the fluorescence of the capillary bead array is detected by a fluorescence reading apparatus, probe beads that capture the fluorescence-labeled target biomolecules and emit fluorescence are detected. However, probe beads that do not capture the fluorescence-labeled target biomolecules are not detected, since no fluorescence is emitted. Consequently, it is very difficult to accurately know the locations and the number of the probe beads that do not emit fluorescence. Therefore, it is also impossible to accurately know the types of the probes that have captured the fluorescence-labeled target biomolecules, since it is impossible to accurately know the order of the probe beads that emit fluorescence. In a capillary bead array, accurately knowing the order of the probe beads, namely, the types of the probes bound to the probe beads, is directly linked to the reliability of a result of biochemical or immunological inspection in which such capillary bead array is used.

Thus, it is an object of the present invention to provide a method for accurately identifying the order of probe beads, namely, the types of the probes bound to the probe beads, by readily and accurately detecting the locations of all the beads in a capillary bead array using a fluorescence reading apparatus. It is also an object of the present invention to significantly improve the reliability of the results of biochemical or immunological inspection in which a capillary bead array is used.

The inventors have found that, as a result of dedicated research, the aforementioned object can be solved, after a multitude of probe beads are arranged inside a capillary, by fluorescent labeling of all the probe beads, by detecting the fluorescence of all the probe beads using a fluorescence reading apparatus, and by determining accurately the locations and the order of all the probe beads in a capillary bead array. They have thus arrived at the present invention.

The present invention is an invention of a method for detecting the locations and the order of all the probe beads in a capillary bead array using a fluorescence reading apparatus. In other words, the present invention is a method for detecting the locations of the probe beads in a capillary bead array, characterized by fluorescent labeling of all the probe beads arranged inside a capillary, or by fluorescent labeling of portions except all the probe beads arranged inside such capillary, to the contrary.

According to the present invention, in a capillary bead array in which a multitude of probe beads are arranged, the locations of all the beads can be detected by the fluorescence reading apparatus, and the order of the probe beads that captured the target biomolecules, namely, the types of the probes that captured the target biomolecules, can be accurately identified simultaneously. Therefore, the accuracy of biochemical or immunological inspection in which a capillary bead array is used can be significantly improved.

### BRIEF DESCRIPTION OF THE DRAWINGS

FIG. 1 shows a bead arrangement (FIG. 1A) and fluorescence development (FIG. 1B) prior to a reaction in a case where the present invention is not carried out.

FIG. 2 shows a bead arrangement (FIG. 2A) and fluorescence development (FIG. 2B) after a reaction in a case where the present invention is not carried out.

FIG. 3 shows a bead arrangement (FIG. 3A) and fluorescence development (FIG. 3B) in a case where the present invention is carried out.

FIG. 4 shows a result image (FIG. 4A) and a bead arrangement (FIG. 4B) in a case where the present invention is not carried out.

FIG. 5 shows result images (FIGS. 5A and 5B) and a bead arrangement (FIG. 5C) in a case where beads are stained by causing a solution of nucleic acid binding dye to flow into a capillary after a reaction with a sample.

FIG. 6 shows result images (FIGS. 6A and 6B) and a bead arrangement (FIG. 6C) in a case where beads are stained by causing a highly concentrated fluorescent dye solution to flow into a capillary.

### DESCRIPTION OF THE PREFERRED EMBODIMENTS

In the present invention, beads are referred to as spherical substances comprising plastic, glass, or the like and having a particle size of several μm to dozens of μm. Specific examples include polystyrene beads, polypropylene beads, and magnetic beads. Their fluorescence development, for example, can be read using a flow cytometer.

In the present invention, as a first method, fluorescent material, such as a solution that includes nucleic acid binding dye, is introduced into the capillary for fluorescent labeling of all the probe beads. As a second method, a solution that includes highly concentrated fluorescent material is introduced into the capillary for fluorescent labeling of portions except all the probe beads.

The fluorescent material may be constituted of organic compounds or inorganic compounds. In the first method, the fluorescent material introduced into the capillary binds to the surface and/or the inside of the probe beads. In this case, the binding refers to physical adsorption, an attractive force due to lyophobic interaction, electrostatic attraction, a covalent bond, a hydrogen bond, or the like. Probes of peptide, protein, nucleic acid, or the like are bound to the surface and/or the inside of the probe beads. Thus, by selecting fluorescent material that has characteristics of specifically binding to the probes of peptide, protein, nucleic acid, or the like, all the probe beads to which the probes are bound can be fluorescence-labeled. Concrete examples include a case where the probe beads, to the surface and/or the inside of which nucleic acid is bound, are arranged inside the capillary. By using organic dye, such as SYTO (trade name), which has the characteristic of binding to nucleic acid, nucleic acid that is bound to the probe beads can be fluorescence-labeled. Consequently, all the probe beads can be fluorescence-labeled. If there are dummy beads to the surface and/or the inside of which the probes are not bound, inside the capillary in addition to the beads, to the surface and/or the inside of which the probes are bound, fluorescent material can be physically adsorbed to the surface and/or the inside of the dummy beads by sufficiently increasing the concentration of the solution of the fluorescent material to be introduced into the capillary. Therefore, the dummy beads can also be fluorescence-labeled in the same manner as the probe beads. As a result, all the beads inside the capillary can be fluorescence-labeled.

In the second method, the highly concentrated fluorescent material introduced into the capillary is allowed to remain in portions except for all the probe beads, and the probe bead portion is outlined. Consequently, the locations and the order of all the beads inside the capillary can be detected. Although the second method is based on an idea contrary to the first method, both methods are effective in detecting the locations and the order of all the beads.

In the present invention, the locations and the order of the probe beads that have captured the target biomolecules can be accurately determined by precisely detecting the locations and the order of all the probe beads using the fluorescence reading apparatus. The probe beads are first arranged inside the capillary formed on soft resin, for example. Although the number of beads to be arranged is not especially limited, several dozens to several hundreds of beads are usually arranged.

Different probes are bound to the surface and/or the inside of the probe beads, respectively. At such time, fluorescent material does not exist on the surface and/or inside the probe beads. A fluorescence image is obtained at this moment by detecting the fluorescence of the capillary in which the probe beads are arranged, using the fluorescence reading apparatus. Upon fluorescence reading, a particular fluorescence wavelength (first fluorescence wavelength) is detected. Fluorescence spots of the probe beads are not detected, since these probe beads do not emit fluorescence at such time.

Then, a solution that includes fluorescence-labeled target biomolecules is introduced from the entrance of the capillary, and the target biomolecules are subjected to a reaction with the probes bound to the surface and/or the inside of the probe beads. In this case, the fluorescent material (first fluorescent material) that is used for fluorescent labeling of the target biomolecules emits the first fluorescence wavelength. The solution is discharged from the exit of the capillary after the reaction has been sufficiently conducted inside the capillary. If necessary, a cleaning fluid is successively introduced from the entrance of the capillary, the capillary and the probe beads are washed, and then the cleaning fluid is discharged from the exit of the capillary. In this case, for all the probe beads, the fluorescence-labeled target biomolecules are captured on the surface and/or the inside of the probe beads bound to the probes that have characteristics of capturing the fluorescence-labeled target biomolecules. A fluorescence image is obtained by detecting the fluorescence of the capillary in which the probe beads are arranged, using the fluorescence reading apparatus. Upon fluorescence reading, detection is conducted using the first fluorescence wavelength.

Only the fluorescence spots of those probe beads that have captured the fluorescence-labeled target biomolecules are detected. Fluorescence spots of other probe beads are not detected. Thus, it is very difficult to accurately know the total number of probe beads arranged inside the capillary or the order of the probe beads that emit fluorescence, using only the then-obtained fluorescence image. This means that it is also impossible to accurately know the types of probes that have captured the fluorescence-labeled target biomolecules.

In the following, the first method is described. A solution that includes nucleic acid binding dye (second fluorescent material) for fluorescent labeling of all the probe beads is successively introduced from the entrance of the capillary from which the solution that includes the fluorescence-labeled target biomolecules has been removed. The solution is discharged from the exit of the capillary after a reaction has been sufficiently conducted. A fluorescence image is obtained by detecting the fluorescence of the capillary in which the probe beads are arranged, using the fluorescence reading apparatus. Upon fluorescence reading, fluorescence wavelength (second fluorescence wavelength) emitted by the second fluorescent material is detected. In this case, fluorescence spots of all the probe beads are detected, since the second fluorescent material is bound to the surfaces and/or the insides of all the probe beads. An analysis is made by overlaying the fluorescence image that includes the fluorescence spots of all the probe beads and the previously obtained fluorescence image that includes only the fluorescence spots of those probe beads that have captured target biomolecules. By using the fluorescence image that includes the fluorescence spots of all the probe beads, it is possible to accurately know the locations and the order of the probe beads in the capillary, said probe beads having captured the fluorescence-labeled biomolecules. Therefore, it is possible to accurately identify the types of probes that have captured the target biomolecules.

In the following, the second method is described. A solution that includes highly concentrated fluorescent material (a second fluorescent material) for fluorescent labeling of portions except all the probe beads is successively introduced from the entrance of the capillary from which the solution that includes the fluorescence-labeled target biomolecules has been removed. A fluorescence image is obtained by detecting the fluorescence of the capillary in which the probe beads are arranged, using the fluorescence reading apparatus. Upon fluorescence reading, the fluorescence wavelength (a second fluorescence wavelength) emitted by the second fluorescent material is detected. In this case, all the probe beads are detected as an outlined fluorescence spot area, since the portions except all the probe beads are stained with the highly concentrated fluorescent material. An analysis is made by overlaying the fluorescence image that includes the outlined fluorescence spot area of all the probe beads and the previously obtained fluorescence image that includes only the fluorescence spots of those probe beads that have captured the target biomolecules. By using a fluorescence image that includes the outlined fluorescence spot area of all the probe beads, it is possible to know accurately the locations and the order of the probe beads in the capillary, said probe beads having captured the fluorescence-labeled biomolecules. Therefore, it is possible to accurately identify the types of probes that have captured the target biomolecules.

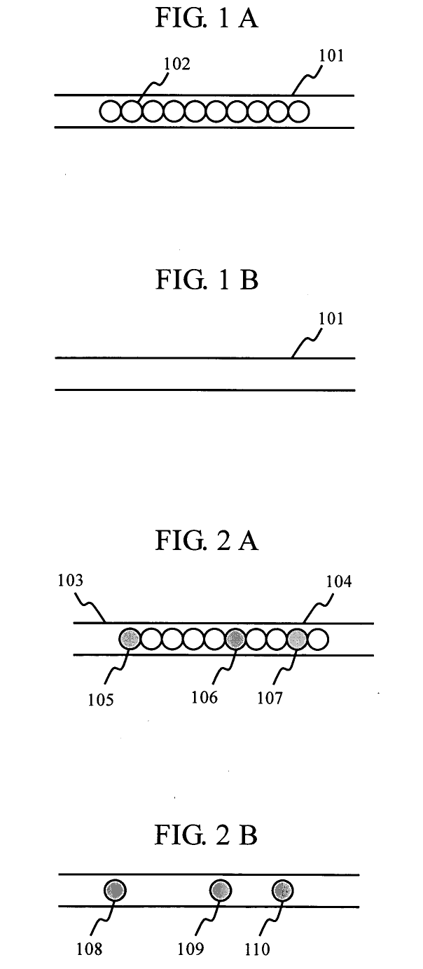

FIGS. 1 to 3 show schematic diagrams of the embodiments of the present invention by which the locations of all the beads in the capillary bead array are detected.

FIGS. 1A and 1B show a bead arrangement and fluorescence development prior to a reaction in a case where the present invention is not carried out. Probe beads 102 are arranged in a capillary 101 formed on soft resin, for example. Although the number of arranged beads is not especially limited, the drawing shows an example where a total of ten probe beads are arranged in a one-dimensional manner for a simple description. Different probes are bound to the ten probe beads, respectively. At this moment, these probe beads 102 do not emit fluorescence (FIG. 1A).

The image shows results obtained by detecting the fluorescence of the capillary 101 in which the probe beads 102 are arranged, using the fluorescence reading apparatus. Upon fluorescence reading, particular fluorescence wavelength (a first fluorescence wavelength) is detected. A fluorescence image of the probe beads 102 is not detected, since these probe beads 102 do not emit fluorescence at this time (FIG. 1B).

FIGS. 2A and 2B show a bead arrangement and fluorescence development after the reaction in a case where the present invention is not carried out. A solution that includes the fluorescence-labeled target biomolecules is introduced from the entrance 103 of the capillary, and the target biomolecules are subjected to a reaction with the probes bound to the probe beads 102. In this case, fluorescent material (a first fluorescent material) that is used for fluorescent labeling of the target biomolecules emits the first fluorescence wavelength. The solution is discharged from the exit 104 of the capillary after the reaction is sufficiently conducted inside the capillary. If necessary, a cleaning fluid is successively introduced from the entrance 103 of the capillary, the inside of the capillary and the probe beads 102 are washed, and then the cleaning fluid is discharged from the exit 104 of the capillary. Probes bound to probe beads 105, 106, and 107 out of the ten probe beads capture the fluorescence-labeled target biomolecules for description (FIG. 2A).

The image shows results obtained by detecting the fluorescence of the capillary in which the probe beads are arranged, using the fluorescence reading apparatus. Upon fluorescence reading, detection is conducted on the basis of the first fluorescence wavelength. Fluorescence spots 108, 109, and 110 corresponding to probe beads 105, 106, and 107 are detected. The fluorescence of other probe beads is not detected. Thus, it is very difficult to accurately know the total number of probe beads arranged inside the capillary and the order of the probe beads that emit fluorescence, using only the fluorescence image shown in the figure. This means that it is also impossible to accurately know the types of the probes that captured the fluorescence-labeled target biomolecules (FIG. 2B).

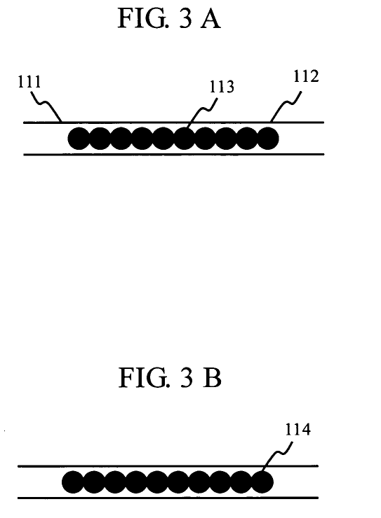

FIGS. 3A and 3B show a bead arrangement and fluorescence development in a case where the present invention is carried out. A solution that includes nucleic acid binding material (second fluorescent material) for fluorescent labeling of all the probe beads is introduced from the entrance 111 of the capillary from which the solution that includes the fluorescence-labeled target biomolecules has been removed. The solution is discharged from the exit 112 of the capillary after a reaction has been sufficiently conducted. All the probe beads 113 are fluorescence-labeled (FIG. 3A).

The image shows results obtained by detecting the fluorescence of the capillary in which the probe beads are arranged, using the fluorescence reading apparatus. Upon fluorescence reading, fluorescence wavelength (a second fluorescence wavelength) emitted by the second fluorescent material is detected. In this case, a fluorescence spot area 114 of all the probe beads is detected, since the second fluorescent material is adsorbed and/or bound to the surface and/or the inside of all the probe beads. The second fluorescence wavelength may be the same as the first fluorescence wavelength for detecting the locations and the order of the probe beads. However, when fluorescence wavelengths are selected such that the second fluorescence wavelength and the first fluorescence wavelength are different, even after the second fluorescent material is bound to all the probe beads, fluorescence reading can be conducted using the first fluorescence wavelength. In other words, the fluorescence image shown in FIG. 2B can be obtained after the fluorescence image shown in FIG. 3B is obtained.

An analysis is made by overlaying the fluorescence image shown in FIG. 2B and the fluorescence image shown in FIG. 3B. Concerning probe beads 108, 109, and 110 whose fluorescence is detected in FIG. 2B, accurate order in the capillary can be determined by using the fluorescence image shown in FIG. 3B. Therefore, it is possible to accurately identify the types of the probes that have captured the target biomolecules.

### Embodiments

In the following, the present invention is described with reference to a comparative example and examples.

### COMPARATIVE EXAMPLE

Hybridization is carried out using maleimide-coated beads. Table 1 shows bead names and characteristics.

TABLE 1

| | Name of oligo | Concentration | |
|---|---|---|---|
| Name | DNA | of oligo DNA | Characteristics |
| V1 beads 5 μM | V1 | 5 μM | V1 is bound to beads coated with |
| | | | maleimide groups. The concentration of |
| | | | V1 upon binding reaction is 5 μM. |
| V1 beads 50 μM | V1 | 50 μM | V1 is bound to beads coated with |
| | | | maleimide groups. The concentration of |
| | | | V1 upon binding reaction is 50 μM. |
| V1 beads 100 μM | V1 | 100 μM | V1 is bound to beads coated with |
| | | | maleimide groups. The concentration of |
| | | | V1 upon binding reaction is 100 μM. |
| V1 beads 200 μM | V1 | 200 μM | V1 is bound to beads coated with |
| | | | maleimide groups. The concentration of |
| | | | V1 upon binding reaction is 200 μM. |
| V2 beads 50 μM | V2 | 50 μM | V2 is bound to beads coated with |
| | | | maleimide groups. The concentration of |
| | | | V1 upon binding reaction is 50 μM. |
| V2 beads 100 μM | V2 | 100 μM | V2 is bound to beads coated with |
| | | | maleimide groups. The concentration of |
| | | | V1 upon binding reaction is 100 μM. |

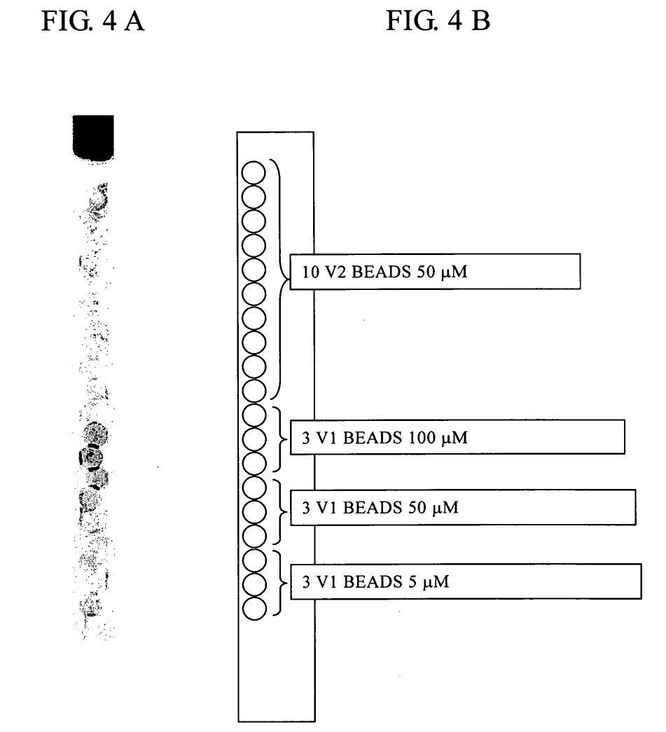

FIG. 4 shows a result image (FIG. 4A) and a bead arrangement (FIG. 4B). From the results of FIG. 4, FIG. 4A shows an image of the situation after a reaction with a sample that includes fluorescence-labeled target DNA has ended. Only three beads near the center have reacted and emit fluorescence. Other beads do not emit fluorescence, so that the confirmation of location is difficult.

### EXAMPLE 1

After the reaction with the sample, the beads are stained by causing a solution of nucleic acid binding dye (SYTO61 (SYTO is a trademark of Molecular Probes Inc.)) to flow into the capillary.

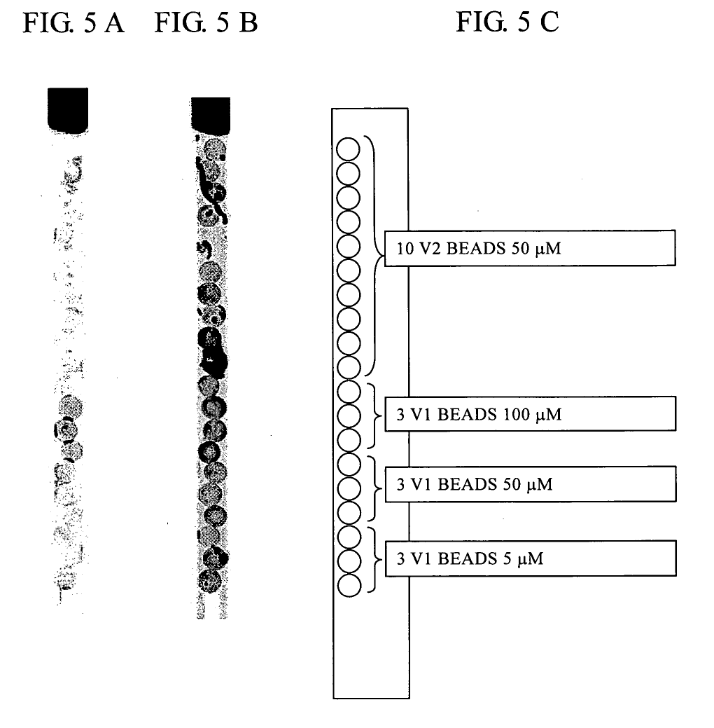

FIG. 5 shows result images (FIGS. 5A and 5B) and a bead arrangement (FIG. 5C). FIG. 5A shows an image of the situation after the reaction experiment with the sample that includes fluorescence-labeled target DNA has ended. Only three beads near the center have reacted. In contrast, FIG. 5B shows an image after the reaction experiment with the sample has ended, and SYTO 61 is further caused to flow and then washed by cleaning fluid finally takes place. From the result of FIG. 5, it is learned that all the beads are stained with SYTO 61 and emit fluorescence. Thus, it is possible to identify the types of reacted beads by comparing the two images of FIGS. 5A and 5B.

### EXAMPLE 2

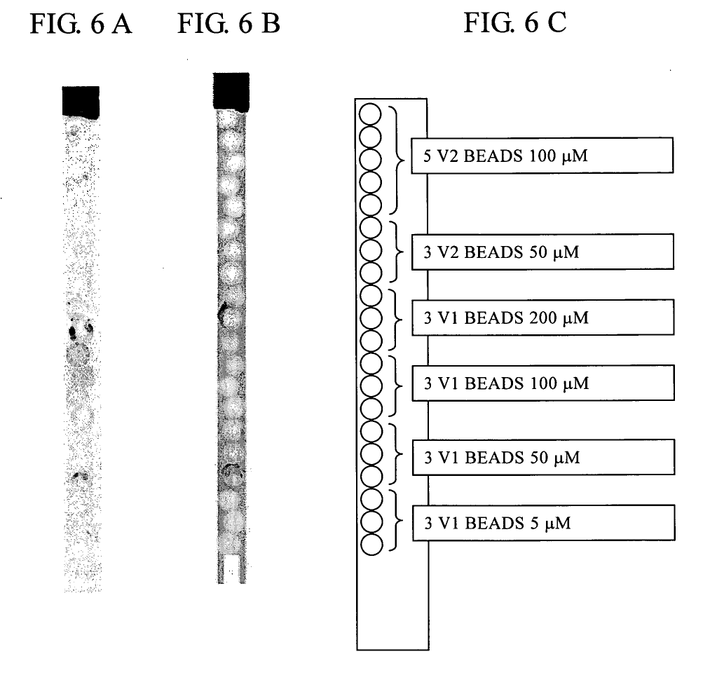

After the reaction with the sample that includes fluorescence-labeled target DNA, the beads are stained by causing a highly concentrated fluorescent dye solution to flow into the capillary. FIG. 6 shows result images (FIGS. 6A and 6B) and a bead arrangement (FIG. 6C). FIG. 6A shows an image of the situation after the reaction experiment with the sample that includes fluorescence-labeled target DNA has ended. Only one bead near the center has reacted. By contrast, FIG. 6B shows an image of the situation after the reaction experiment with the sample has ended, and the highly concentrated fluorescent dye solution is caused to flow into the capillary. The beads are shown as an outlined image, since the solution emits intense fluorescence when the highly concentrated fluorescent dye solution is caused to flow. From the result of FIG. 6, it is learned that all the beads can be shown as an outlined image. Thus, it is possible to identify the types of reacted beads by comparing FIGS. 6A and 6B.

As in the present invention, the utility of the capillary bead array can be improved by identifying the locations and the order of all the beads. Moreover, according to the present invention, the utility of the capillary bead array can be improved, since a conventional fluorescence reading apparatus can be used without modification.

Therefore, the reliability of the results of biochemical or immunological inspection in which a capillary bead array is used can be significantly improved.

1. A method for detecting the locations of probe beads in a capillary bead array in which a multitude of probe beads are arranged in a capillary formed on a substrate, wherein the multitude of probe beads have probes bound to the surfaces and/or the insides thereof, the probes having characteristics such that they capture target biomolecules, said method comprising the steps of causing a solution that includes fluorescent dye that has characteristics such that it specifically binds to the probes to pass inside the capillary, and detecting the locations of all the beads arranged inside the capillary using a fluorescence reading apparatus by determining a portion of fluorescence development as a bead location.

2. A method for detecting the locations of probe beads in a capillary bead array in which a multitude of probe beads are arranged in a capillary formed on a substrate, wherein the multitude of probe beads have probes bound to the surfaces and/or the insides thereof, the probes having characteristics such that they capture target biomolecules, said method comprising the steps of causing a solution that includes highly concentrated fluorescent dye to pass inside the capillary, and detecting the locations of all the beads arranged inside the capillary using a fluorescence reading apparatus by determining a portion of no fluorescence development as a bead location.

3. The method for detecting the locations of probe beads in a capillary bead array according to claim 1, comprising introducing a solution that includes fluorescence-labeled target biomolecules into the capillary, capturing the biomolecules on the probe beads, wherein the probe beads have probes bound to the surfaces and/or the insides thereof, the probes having characteristics such that they capture the target biomolecules, obtaining, using the fluorescence reading apparatus, a fluorescence image that includes only the fluorescence spots of those of the multitude of the probe beads arranged inside the capillary of the capillary bead array that have captured the target biomolecules via the probes bound to the surfaces and/or the insides of the probe beads, comparing the fluorescence image with a fluorescence image that includes the fluorescence spot area of all the probe beads, and determining the locations and the order of the probe beads that have captured the target biomolecules inside the capillary bead array.

4. The method for detecting the locations of probe beads in a capillary bead array according to claim 2, comprising introducing a solution that includes fluorescence-labeled target biomolecules into the capillary, capturing the biomolecules on the probe beads, wherein the probe beads have probes bound to the surfaces and/or the insides thereof, the probes having characteristics such that they capture the target biomolecules, obtaining, using the fluorescence reading apparatus, a fluorescence image that includes only the fluorescence spots of those of the multitude of the probe beads arranged inside the capillary of the capillary bead array that have captured the target biomolecules via the probes bound to the surfaces and/or the insides of the probe beads, comparing the fluorescence image with a fluorescence image that includes the spot area of all the probe beads that have no fluorescence development, and determining the locations and the order of the probe beads that have captured the target biomolecules inside the capillary bead array.

5. The method for detecting the locations of probe beads in a capillary bead array according to claim 1, wherein the step of detecting the locations of all the beads using the fluorescence reading apparatus is carried out following the steps of introducing the solution that includes fluorescence-labeled target biomolecules into the capillary, capturing the biomolecules on the probe beads, wherein the probe beads have the probes bound to the surfaces and/or the insides thereof, the probes having such characteristics that they capture the target biomolecules, and obtaining, using the fluorescence reading apparatus, a fluorescence image that includes only the fluorescence spots of those of all the multitude of the probe beads arranged inside the capillary of the capillary bead array that have captured the target biomolecules via the probes bound to the surfaces and/or the insides of the probe beads.

6. The method for detecting the locations of probe beads in a capillary bead array according to claim 2, wherein the step of detecting the locations of all the beads using the fluorescence reading apparatus is carried out following the steps of introducing the solution that includes fluorescence-labeled target biomolecules into the capillary, capturing the biomolecules on the probe beads, wherein the probe beads have the probes bound to the surfaces and/or the insides thereof, the probes having such characteristics that they capture the target biomolecules, and obtaining, using the fluorescence reading apparatus, a fluorescence image that includes only the fluorescence spots of those of all the multitude of the probe beads arranged inside the capillary of the capillary bead array that have captured the target biomolecules via the probes bound to the surfaces and/or the insides of the probe beads.

7. The method for detecting the locations of probe beads in a capillary bead array according to claim 3, wherein the step of detecting the locations of all the beads using the fluorescence reading apparatus is carried out following the steps of introducing the solution that includes fluorescence-labeled target biomolecules into the capillary, capturing the biomolecules on the probe beads, wherein the probe beads have the probes bound to the surfaces and/or the insides thereof, the probes having such characteristics that they capture the target biomolecules, and obtaining, using the fluorescence reading apparatus, a fluorescence image that includes only the fluorescence spots of those of all the multitude of the probe beads arranged inside the capillary of the capillary bead array that have captured the target biomolecules via the probes bound to the surfaces and/or the insides of the probe beads.

8. The method for detecting the locations of probe beads in a capillary bead array according to claim 1, wherein said comparison of the two fluorescence images is made by overlaying two images.

9. The method for detecting the locations of probe beads in a capillary bead array according to claim 2, wherein said comparison of the two fluorescence images is made by overlaying two images.

10. The method for detecting the locations of probe beads in a capillary bead array according to claim 3, wherein said comparison of the two fluorescence images is made by overlaying two images.
